(19) United States
(12) Patent Application Publication
(10) Pub. No.: US 2022/0354870 A1
(43) Pub. Date: Nov. 10, 2022

(54) READY-TO-USE BUPIVISONE PLUS LIQUID FORMULATION

(71) Applicant: LEITERS, Inc., Englewood, CO (US)

(72) Inventors: Dominic Ventura, Wilmington, NC (US); Dona Wickramaratne, Aurora, CO (US); Chris Zuccarelli, Hampton, NJ (US); Glenn HUTH, Barberton, OH (US); Mark THOMAS, Akron, OH (US)

(73) Assignee: LEITERS, Inc., Englewood, CO (US)

(21) Appl. No.: 17/720,556

(22) Filed: Apr. 14, 2022

Related U.S. Application Data
(60) Provisional application No. 63/174,858, filed on Apr. 14, 2021.
(60) Provisional application No. 63/292,096, filed on Dec. 21, 2021.

(51) Int. Cl. A61K 31/573; A61K 31/445; A61K 31/137; A61K 47/02; A61K 47/06

(52) U.S. Cl. CPC A61K 31/573; A61K 31/445; A61K 31/137; A61K 47/02; A61K 47/06

(57) ABSTRACT

The present disclosure provides a ready-to-use liquid formulation comprising bupivacaine hydrochloride, dexamethasone sodium phosphate and epinephrine (collectively referred to as Bupivisone Plus), and methods for preparing, packaging, storing and using the same.

### CROSS-REFERENCE TO RELATED APPLICATIONS

This application claims benefit under 35 U.S.C. § 119(e) of the U.S. Provisional Application No. 63/174,858, filed Apr. 14, 2021 and U.S. Provisional Application No. 63/292,096, filed Dec. 21, 2021, the contents of both of which are incorporated herein by reference in their entireties.

### TECHNICAL FIELD

Disclosed herein is a ready-to-use liquid formulation comprising bupivacaine hydrochloride, dexamethasone sodium phosphate and epinephrine (collectively referred to as Bupivisone Plus). Also disclosed herein is a process for preparing the Bupivisone Plus, as well as method for packaging and storing the same.

### BACKGROUND

The surgical reconstruction or replacement of a joint, known as arthroplasty, may be necessary to alleviate pain associated with osteoarthritis. A problem associated with arthroplasty is the management of post-operative pain. Indeed, it is generally recognized that failure to control pain adequately following an arthroplasty procedure may hinder physiotherapy and increase anxiety.

Bupivisone Plus may be used as a local anesthetic during an arthroplasty procedure. In fact, some hospitals use a Bupivisone Plus solution during knee arthroplasty to improve immediate post-operative pain relief. Generally, Bupivisone Plus is premixed by a pharmacy and is limited in terms of its usable shelf-life as it remains stable for only about twenty-four hours.

In addition to the limited stability and shelf-life of Bupivisone Plus, pharmacy preparation for a Bupivisone Plus-containing solution has the potential for microbial contamination and/or errors associated with compounding. Extemporaneously preparing Bupivisone Plus from commercial vials of each active is also problematic. The problem is that the pH of each individual active vial solution can vary slightly from lot to lot due to manufacturing variations as well the age of the commercial product. Assurance of Bupivisone Plus stability is pH dependent and thus can only be assured through exact control of the final Bupivisone Plus solution pH. Extemporaneous preparations of Bupivisone Plus from commercial vials of each active cannot be controlled for final pH and this cannot assu2re consistent stability (storage time or beyond use date) for each preparation. The criticality of pH is due to epinephrine being susceptible to oxidation at higher pH's whereas dexamethasone is susceptible to precipitation and formation of objectional particulates at lower pH's (secondarily dexamethasone is susceptible to hydrolysis at low and high pH). As such, there are at least three drawbacks with the current extemporaneously prepared Bupivisone Plus preparations:
a) the process is expensive, increasing the manufacturing cost per syringe using available separate vials of each active ingredient;
b) the admixture and dilution require numerous aseptic manipulations on the part of the health care giver which could lead to sterility compromises which may not be detected especially for extemporaneously prepared solutions in hospital settings which do not generally undergo vigorous GMP type batch testing, such as potency, pH and impurities on a batch basis; and
c) the product pH is variable due to the inherent pH allowed for each individual active currently allowed by USP/FDA product specifications resulting in variable final admixture pH (e.g. the allowed pH range for Bupivicaine Hydrochloride Injection, USP is 4.0 to 6.5) and the associated variable stability (Expiry period).

Thus, there is a need for a pH controlled compounded formulation containing Bupivisone Plus that overcomes the problems of the extemporaneously prepared Bupivisone Plus solutions to ensure that patients receive a Bupivisone Plus injection with assured shelf-stability, potency, and purity at the time of the injection. Of particular concern is the propensity of the epinephrine to degrade by oxidation which can limit the stability of the formula. The present disclosure addresses some of these needs.

### SUMMARY

Inventors have discovered inter alia that a liquid formulation comprising bupivacaine hydrochloride (HCl), dexamethasone sodium phosphate and epinephrine (e.g., in form of epinephrine hydrochloride) is stable over time when the pH of the formulation is in a range from about 3.2 to about 4.4. Accordingly, in one aspect, provided herein is a ready-to-use liquid formulation comprising bupivacaine HCl, dexamethasone sodium phosphate and epinephrine having a pH in a range from about 3.5 to about 4.1. In some embodiments, the formulation has a pH in a range from about 3.7 to about 3.9.

In some embodiments, the formulation is substantially free of antimicrobial preservatives, antifungal preservatives, and/or antiprotozoan preservatives. For example, the formulation is substantially free of antimicrobial preservative, such as methylparaben.

### BRIEF DESCRIPTION OF THE DRAWINGS

FIG. 1 is a line graph showing the potency of Bupivacaine HCI in some exemplary formulations of the disclosure over various time points.

FIG. 2 is a line graph showing the potency of Dexamethasone Phosphate in some exemplary formulations of the disclosure over various time points.

FIG. 3 is a line graph showing the potency of Epinephrine in some exemplary formulations of the disclosure over various time points.

FIG. 4 is a line graph showing the pH trend of Bupivacaine HCI, Dexamethasone Phosphate and Epinephrine in some exemplary formulations of the disclosure over various time points.

### DETAILED DESCRIPTION

Generally, each of bupivacaine hydrochloride, epinephrine and dexamethasone sodium in the formulation have a potency of at least about 90% after storage for about 90 days at a temperature of about 5° (degrees) C. and a subsequent storage of about 25° C. for a minimum of about 72 hours or any kinetic temperature equivalent storage.

As the ready-to-use formulation is stable, the amount of impurities remains low, even after long term storage. The amount of any impurities in the formulation is lower than that permitted by the ICH/FDA guidelines such as ICH Topic Q 3 B (R2) Impurities in New Drug Products: GUIDANCE ON IMPURITIES IN NEW DRUG PRODUCTS (CPMP/ICH/2738/99), content of which is incorporated herein by reference in its entirety. For example, the formulation does not comprise an impurity exceeding ICH/FDA guidelines after storage for about 90 days at a temperature of about 5° C. and a subsequent storage of about 25° C. for a minimum of about 72 hours or any kinetic temperature equivalent storage. In some embodiments, an impurity in the formulation is below about 1%, below about 0.5%, or below about 0.25% after storage for about 90 days at a temperature of about 5° C. and a subsequent storage of about 25° C. for a minimum of about 72 hours or any kinetic temperature equivalent storage.

The amount of bupivacaine HCl, dexamethasone sodium phosphate and epinephrine in the formulation can vary. For example, the formulation can comprise bupivacaine HCl in an amount from about 3.25 mg/mL to about 4.25 mg/mL. In some embodiments, the formulation comprises bupivacaine hydrochloride in an amount from about 3.5 mg/mL to about 4 mg/mL. Preferably, the formulation comprises bupivacaine hydrochloride in an amount of about 3.75 mg/mL. The desired amount of bupivacaine HCl in the formulation depends on physician preference.

Similarly, the amount of epinephrine in the formulation can vary. For example, the formulation can comprise epinephrine hydrochloride in an amount from about 4.5 mcg/mL to about 5.5 mcg/mL, based on an epinephrine free base. In some embodiments, the formulation comprises epinephrine hydrochloride in an amount from about 4.75 mcg/mL to about 5.25 mcg/mL, based on an epinephrine free base. Preferably, the formulation comprises epinephrine hydrochloride in an amount of about 5 mcg/mL, based on an epinephrine free base. The desired amount of epinephrine hydrochloride in the formulation depends on physician preference.

The amount of dexamethasone sodium phosphate in the formulation can also vary. For exmaple, the formulation can comprise dexamethasone sodium phosphate in an amount from about 0.05 mg/mL to about 0.15 mg/mL. In some embodiments, the formulation comprises dexamethasone sodium phosphate in an amount from about 0.075 mg/mL to about 0.125 mg/mL. Preferably, the formulation comprises dexamethasone sodium phosphate in an amount of about 0.1 mg/mL. The desired amount of dexamethasone sodium phosphate in the formulation depends on physician preference.

In addition to the active agents, i.e., bupivacaine HCl, dexamethasone sodium phosphate and epinephrine, the formulation can comprise one or more pH adjusting agents for adjusting the pH of the formulation to a desired pH, e.g., pH in a range from about 3.5 to about 4.1, preferably in a range from about 3.7 to about 3.9. The desired pH of the formulation depends on factors such as degradation of epinephrine and/or dexamethasone as well as an acceptable pH range for a parenteral intended for injection or infusion. Exemplary pH adjusting agents can include, for example, acids such as organic acids and mineral acids, bases such as organic bases and hydroxides of alkaline metals and alkaline earth metals. Exemplary organic acids can include, for example, acetic, lactic, formic, citric, oxalic, and uric acids. Exemplary mineral acids can include, for example, hydrochloric, nitric, phosphoric, sulphuric, boric, hydrofluoric, hydrobromic and perchloric acids. Exemplary organic bases can include, for example, pyridine, methylamine, imidazole, benzimidazole, histidine, phosphazene, and hydroxides of cations. Exemplary hydroxides of alkali metal and alkaline earth metals can include, for example, potassium hydroxide (KOH), barium hydroxide (Ba(OH)₂), caesium hydroxide (CsOH), sodium hydroxide (NaOH), strontium hydroxide (Sr(OH)₂), calcium hydroxide (Ca(OH)₂), lithium hydroxide (LiOH), and rubidium hydroxide (RbOH). In some embodiments, the pH adjusting agent is hydrochloric acid, sodium hydroxide or a combination thereof.

The formulation can comprise one or more pharmaceutically acceptable excipients and/or diluents. For example, such excipients include salts, and other excipients. Accordingly, in some embodiments, the formulation comprises a salt. Exemplary salts include alkaline earth metal salts such as sodium, potassium, and calcium. Counter ions include chloride and phosphate. For example, the formulation can comprise a salt selected from the group consisting of sodium chloride, potassium chloride, magnesium chloride, calcium chloride, and potassium phosphate. In some embodiments, the salt is sodium chloride.

When present, the amount of the salt in the formulation can be varied. For example, the formulation can comprise a salt in an amount from about 7.7 mg/mL to about 8.7 mg/mL. In some embodiments, the formulation comprises the salt in an amount from about 7.95 mg/mL to about 8.45 mg/mL. Preferably, the formulation comprises the salt in an amount of about 8.2mg/mL. The desired amount of salt, when present in the formulation, depends on the actual concentration of actives and other excepients which could influence the final osmolarity of the solution.

In some embodiments, the formulation comprises sodium chloride. For example, the formulation comprises sodium chloride in an amount from about 7.7 mg/mL to about 8.7 mg/mL. In some embodiments, the formulation comprises sodium chloride in an amount from about 7.95 mg/mL to about 8.45 mg/mL. In further embodiments, the formulation comprises sodium chloride in an amount of about 8.2 mg/mL.

The formulation can also comprise an antioxidant. Exemplary antioxidants include, for example, sodium metabisulfite, sodium sulfite, sodium bisulfate, sodium thiosulfate, vitamin C (ascorbic acid), vitamin E, vitamin A, B vitamins (e.g. vitamin B₆), flavonoids, selenium, carotenoids (e.g. beta-carotene). Additional exemplary antioxidants include, for example, propyl, octyl and dodecyl esters of gallic acid, butylated hydroxyanisole (BHA), butylated hydroxytoluene (BHT), nordihydroguairetic acid, and alkylated parabens such as methylparaben and propylparaben. In some embodiments, the antioxidant is sodium metabisulfite.

Like the other components in the formulation, the amount of the antioxidant can be varied as needed. For example, the formulation can comprise an antioxidant in an amount from about 18 mcg/mL to about 28 mcg/mL. In some embodiments, the formulation comprises an antioxidant in an amount from about 20 mcg/mL to about 26 mcg/mL. Preferably, the formulation comprises an antioxidant in an amount of about 23 mcg/mL. The desired amount of antioxidant depends on factors such as the concentration of epinephrine, storage temperature, oxygen ingress and pH.

In some embodiments, the formulation comprises sodium metabisulfite. For example, the formulation comprises sodium metabisulfite in an amount from about 18mcg/mL to about 28 mcg/mL. In some embodiments, the formulation comprises sodium metabisulfite in an amount from about 20 mcg/mL to about 26 mcg/mL. Preferably, the formulation comprises sodium metabisulfite in an amount of about 23 mcg/mL.

In some embodiments, the formulation can further comprise one or more preservatives. Exemplary preservatives include, for example, antioxidants, chelating agents, antimicrobial preservatives, antifungal preservatives, antiprotozoan preservatives, alcohol preservatives, acidic preservatives, and other preservatives.

In some embodiments, the preservative can be an antimicrobial agent. The antimicrobial agent can be, for example, substances which have fungicidal activity (fungicidal agents) and/or substances which have bactericidal activity (bactericidal agents). Exemplary antimicrobial agents include, but are not limited to, parabens, isothiazolinone, phenolics, acidic preservatives, halogenated compounds, quarternia, and alcohol. Exemplary parabens can include, but are not limited to, parabens and paraben salts. Exemplary isothiazolinones can include, but are not limited to, methylchloroisothiazolinone, methylisothiazolinone, benzisothiazolinone ProClin 150, ProClin 200, ProClin 300, and ProClin 950. Exemplary phenolic types can include, but are not limited to, phenoxyethanol, benzyl alcohol, and phenethyl alcohol. Exemplary acidic preservatives can include, but are not limited to, dehydroacetic acid, benzoic acid, sorbic acid, salicylic acid, formic acid, propionic acid. Exemplary halogenated compounds can include, but are not limited to, 2-bromo-2-nitropropane-1, 3-diol, chloroacetamide, chlorobutanol, chloroxylenol, chlorphenesin, dichlorobenzyl alcohol, iodopropynyl butylcarbamate, methyldibromo glutaronitrile. Exemplary quaternia can include, but are not limited to, benzalkonium chloride, benzethonium chloride, chlorhexidine, hexamidine diisethionate, and polyaminopropyl biguanide. Exemplary alcohols can include, but are not limited to, ethyl alcohol and isopropyl alcohol. Examples thereof include, but are not limited to, triazine antimicrobial agents, thiazole bactericidal agents (for example, benzisothiazolone etc.), pyrithione, pyridine bactericidal agents (for example, 1-hydroxy pyridine-2-thiosodium etc.), 2-phenoxyethanol, and the like. In some embodiments, the antimicrobial agent is methylparaben. In other embodiments, the preferred antimicrobial is propylparaben.

In some further embodiments, the formulation comprises methylparaben and propylparaben.

In some embodiments, the formulation can further comprise a phenolic preservative. For example, the formulation can comprise a phenolic preservative such as phenoxyethanol, benzyl alcohol or phenethyl alcohol. In some embodiments, the formulation comprises benzyl alcohol. In some further embodiments, the formulation comprises methylparaben and benzyl alcohol.

The amount of the antimicrobial agent can be varied as needed. For example, the formulation can comprise an antimicrobial agent in an amount from about 0.65 mg/mL to about 0.85 mg/mL. In some embodiments, the formulation comprises an antimicrobial agent in an amount from about 0.7 mg/mL to about 0.8 mg/mL. In other embodiments, the formulation comprises an antimicrobial agent in an amount of 0.75 mg/mL.

In some embodiments, the formulation comprises methylparaben. For example, the formulation can comprise methylparaben in an amount from about 0.65 mg/mL to about 0.85 mg/mL. In some embodiments, the formulation comprises an antimicrobial agent in an amount from about 0.7 mg/mL to about 0.8 mg/mL. Preferably, the formulation comprises an antimicrobial agent in an amount of 0.75 mg/mL.

In some embodiments, the formulation comprises bupivacaine hydrochloride, epinephrine hydrochloride, dexamethasone sodium phosphate, a salt, an antioxidant, an antimicrobial agent, and a pH adjusting agent to obtain a pH of from about 3.5 to about 4.1, and, more specifically, a pH from about 3.7 to about 3.9. For example, the formulation comprises bupivacaine hydrochloride, epinephrine hydrochloride, dexamethasone sodium phosphate, sodium chloride, sodium metabisulfite, methylparaben, and a pH adjusting agent to obtain a pH of from about 3.5 to about 4.1, preferably a pH from about 3.7 to about 3.9.

In some embodiments, the formulation comprises bupivacaine hydrochloride, epinephrine hydrochloride, dexamethasone sodium phosphate, a salt, and a pH adjusting agent to obtain a pH of from about 3.5 to about 4.1, and, more specifically, a pH from about 3.7 to about 3.9. For example, the formulation comprises bupivacaine hydrochloride, epinephrine hydrochloride, dexamethasone sodium phosphate, sodium chloride, and a pH adjusting agent to obtain a pH of from about 3.5 to about 4.1, preferably a pH from about 3.7 to about 3.9.

In some other embodiments, the formulation comprises bupivacaine hydrochloride, epinephrine hydrochloride, dexamethasone sodium phosphate, a salt, an antioxidant, and a pH adjusting agent to obtain a pH of from about 3.5 to about 4.1, and, more specifically, a pH from about 3.7 to about 3.9. For example, the formulation comprises bupivacaine hydrochloride, epinephrine hydrochloride, dexamethasone sodium phosphate, sodium chloride, sodium metabisulfite, and a pH adjusting agent to obtain a pH of from about 3.5 to about 4.1, preferably a pH from about 3.7 to about 3.9.

In still further embodiments, the formulation comprises bupivacaine hydrochloride in an amount of about 3.75 mg/mL; epinephrine hydrochloride in an amount of about 5 mcg/mL, based on an epinephrine free base; dexamethasone sodium phosphate in an amount of about 0.1 mg/mL; sodium chloride in an amount of about 8.2 mg/mL; a sufficient amount of sterile water for injection; and a sufficient amount of a pH adjusting agent to obtain a pH of from about 3.7 to about 3.9.

In still further embodiments, the formulation comprises bupivacaine hydrochloride in an amount of about 3.75 mg/mL; epinephrine hydrochloride in an amount of about 5 mcg/mL, based on an epinephrine free base; dexamethasone sodium phosphate in an amount of about 0.1 mg/mL; sodium chloride in an amount of about 8.2 mg/mL; sodium metabisulfite in an amount of about 23 mcg/mL; a sufficient amount of sterile water for injection; and a sufficient amount of a pH adjusting agent to obtain a pH of from about 3.7 to about 3.9.

In still further embodiments, the formulation comprises bupivacaine hydrochloride in an amount of about 3.75 mg/mL; epinephrine hydrochloride in an amount of about 5 mcg/mL, based on an epinephrine free base; dexamethasone sodium phosphate in an amount of about 0.1 mg/mL; sodium chloride in an amount of about 8.2 mg/mL; sodium metabisulfite in an amount of about 23 mcg/mL; methylparaben in an amount of 0.75 mg/mL; a sufficient amount of sterile water for injection; and a sufficient amount of a pH adjusting agent to obtain a pH of from about 3.7 to about 3.9.

In some embodiments, the formulation is substantially free of preservatives. For example, the formulation is substantially free of antimicrobial preservatives, antifungal preservatives, and/or antiprotozoan preservatives. In some embodiments, the formulation is substantially free of methylparaben.

As used herein, “substantially free” means that the indicated component, e,g., a preservative is present in an amount less than about 5% (v/v, w/v, or w/w) of formulation. For example, the indicated component, e,g., a preservative, such as an antimicrobial preservative, antifungal preservative, and/or antiprotozoan preservative, e.g., methylparaben is present in an amount less than about 4.5%, or less than about 4%, or less than about 3.5%, or less than about 3%, or less than about 2.5%, or less than about 2%, or less than about 1.5%, or less than about 1%, or less than about 0.5%, or less than 0.25% (v/v, w/v, or w/w), or less than 0.20% (v/v, w/v, or w/w), or less than 0.15% (v/v, w/v, or w/w), or less than 0.05% (v/v, w/v, or w/w), or less than 0.01% (v/v, w/v, or w/w) of the formulation. In some embodiments, the indicated component, e.g., a preservative, such as an antimicrobial preservative, antifungal preservative, and/or antiprotozoan preservative, e.g., methylparaben is present in an undetectable amount. For example, the indicated component, e.g., a preservative, such as an antimicrobial preservative, antifungal preservative, and/or antiprotozoan preservative, e.g., methylparaben is present in an amount that is undetectable by HPLC, gas chromatography or mass spectrometry used for detecting the indicated component.

In some embodiments, the formulation comprises bupivacaine hydrochloride, epinephrine hydrochloride, dexamethasone sodium phosphate, a salt, an antioxidant, and a pH adjusting agent to obtain a pH of from about 3.5 to about 4.1 (e.g., a pH from about 3.7 to about 3.9), and wherein the formulation is substantially free of preservatives, e,g., an antimicrobial preservative, antifungal preservative, and/or antiprotozoan preservative, such as methylparaben. For example, the formulation comprises bupivacaine hydrochloride, epinephrine hydrochloride, dexamethasone sodium phosphate, sodium chloride, sodium metabisulfite, and a pH adjusting agent to obtain a pH of from about 3.5 to about 4.1 (e.g., a pH from about 3.7 to about 3.9), and wherein the formulation is substantially free of preservatives.

In some embodiments, the formulation comprises bupivacaine hydrochloride, epinephrine hydrochloride, dexamethasone sodium phosphate, a salt, and a pH adjusting agent to obtain a pH of from about 3.5 to about 4.1 (e.g., a pH from about 3.7 to about 3.9), and the formulation is substantially free of preservatives, e.g., antimicrobial preservatives, antifungal preservatives, and/or antiprotozoan preservative, such as methylparaben. For example, the formulation comprises bupivacaine hydrochloride, epinephrine hydrochloride, dexamethasone sodium phosphate, sodium chloride, and a pH adjusting agent to obtain a pH of from about 3.5 to about 4.1 (e.g., a pH from about 3.7 to about 3.9), and wherein the formulation is substantially free of preservatives.

In some other embodiments, the formulation comprises bupivacaine hydrochloride, epinephrine hydrochloride, dexamethasone sodium phosphate, a salt, an antioxidant, and a pH adjusting agent to obtain a pH of from about 3.5 to about 4.1 (e.g., a pH from about 3.7 to about 3.9), and wherein the formulation is substantially free of preservatives, e.g., antimicrobial preservatives, antifungal preservatives, and/or antiprotozoan preservatives, such as methylparaben. For example, the formulation comprises bupivacaine hydrochloride, epinephrine hydrochloride, dexamethasone sodium phosphate, sodium chloride, sodium metabisulfite, and a pH adjusting agent to obtain a pH of from about 3.5 to about 4.1 (e.g., a pH from about 3.7 to about 3.9), and wherein the formulation is substantially free of preservatives.

In still further embodiments, the formulation comprises bupivacaine hydrochloride in an amount of about 3.75 mg/mL; epinephrine hydrochloride in an amount of about 5 mcg/mL, based on an epinephrine free base; dexamethasone sodium phosphate in an amount of about 0.1 mg/mL; sodium chloride in an amount of about 8.2 mg/mL; a sufficient amount of sterile water for injection; and a sufficient amount of a pH adjusting agent to obtain a pH of from about 3.7 to about 3.9, and wherein the formulation is substantially free of preservatives, e.g., antimicrobial preservatives, antifungal preservatives, and/or antiprotozoan preservatives, such as methylparaben.

In still further embodiments, the formulation comprises bupivacaine hydrochloride in an amount of about 3.75 mg/mL; epinephrine hydrochloride in an amount of about 5 mcg/mL, based on an epinephrine free base; dexamethasone sodium phosphate in an amount of about 0.1 mg/mL; sodium chloride in an amount of about 8.2 mg/mL; sodium metabisulfite in an amount of about 23 mcg/mL; a sufficient amount of sterile water for injection; and a sufficient amount of a pH adjusting agent to obtain a pH of from about 3.7 to about 3.9, and wherein the formulation is substantially free of preservatives, e.g., antimicrobial preservatives, antifungal preservatives, and/or antiprotozoan preservatives, such as methylparaben.

The formulation described herein can be comprised in a container. Accordingly, in another aspect, provided herein is a container comprising a formulation described herein. The liquid formulation may be freeze-dried to permit enhanced ease for packaging, transporting and storage of the containers comprising the formulation.

The ready-to-use liquid formulation described herein may be packaged in a pre-filled container, such as, for example, a glass vial closed with a stopper or other closure that includes a septum through which a hypodermic needle may be inserted to withdraw the formulation, or may be packaged in a pre-filled syringe or other pre-filled container suitable for injection (e.g., subcutaneous injection) or infusion. Examples of such containers include, without limitation, vials, syringes, ampoules, bottles, cartridges, and pouches. Preferably the containers are each single-use prefilled syringes, which may suitably be formed of glass or a polymeric material such as a cyclic olefin polymers or acrylonitrile butadiene styrene (ABS), polycarbonate (PC), polyoxymethylene (POM), polystyrene (PS), polybutylene terephthalate (PBT), polypropylene (PP), polyethylene (PE), polyamide (PA), thermoplastic elastomer (TPE), and their combinations. The barrels of such syringes are operated with an elastomer plunger which can be urged along the barrel to eject liquid content via a needle connected thereto. In some embodiments of the invention, each syringe includes a needle affixed thereto. In some embodiments, the ready-to-use liquid formulation as disclosed herein is contained within a pre-filled container selected from the group consisting of: a syringe (e.g., a single or double barreled syringe), a pen injector, a sealed vial (e.g., a dual chamber vial), a cassette, and a pump device (e.g., an on-body patch pump, a tethered pump or an osmotic pump)

In some embodiments, the container containing a formulation described herein is a light sensitive container. For example, the container can be a light-sensitive container having a light transmission of less than about 5%, e.g., less than about 4.5%, less than about 4%, less than about 3.5%, less than about 3%, less than about 2.5%, less than about 2%, less than about 1.5% or less than about 1% at any wavelength between about 290 nm and about 450 nm. In some embodiments, the light-sensitive container comprises an amber-colored film or an amber-colored plastic.

In some embodiments, the container containing a formulation described herein is hermetically sealed. For example, the container containing a formulation described herein is hermetically sealed to protect the formulation from oxygen intrusion.

In some embodiments, the container comprising a formulation described herein is a container labeled for use. For example, the container is labeled for therapeutic use.

The formulations of the present disclosure can be prepared as unit dosage forms in a pre-filled container. For example, the formulations of the present disclosure can be prepared as unit dosage forms in pre-filled containers such as a unit dosage per vial, cartridge or other pre-filled container (e.g., pre-filled syringe or disposable pen) which may contain about 10 ml, about 15 ml, about 20 ml, about 25 ml, about 30 ml, about 35 ml, about 40 ml, about 45 ml, about 50 ml, about 55 ml, about 60 ml, about 65 ml, about 70 ml, about 75 ml, about 80 ml, about 85 ml, about 90 ml, about 95 ml or about 100 ml of a ready-to-use liquid formulation described herein.

The formulations of the disclosure are stable over long-term storage, i.e., formulations have long-term stability. The term “long-term storage” or “long term stability” is understood to mean that the formulation can be stored for about one week, for about two weeks, for about three weeks, for about one month, for about two months, for about three months, for about six months or more. Generally speaking, the terms “long term storage” and “long term stability” further include stable storage durations that are at least comparable to or better that the stable shelf typically required for currently available formulations, without losses in stability that would render the formulation unsuitable for its intended pharmaceutical application. Long-term storage is also understood to mean that the pharmaceutical composition is stored either as a liquid at about 2° C. to about 8° C., or is frozen, e.g., at about −20° C., or colder. It is also contemplated that the composition can be frozen and thawed more than once.

The term “stable” with respect to long-term storage is understood to mean that an active ingredient, e.g., bupivacaine hydrochloride, epinephrine and/or dexamethasone sodium in the formulation does not lose more than about 20% of its activity relative to activity of the composition at the beginning of storage. For example, an active ingredient, e.g., bupivacaine hydrochloride, epinephrine and/or dexamethasone sodium in the formulation does not lose more than about 15% of its activity relative to activity of the composition at the beginning of storage. In some embodiments, an active ingredient, e.g., bupivacaine hydrochloride, epinephrine and/or dexamethasone sodium in the formulation does not lose more than about 10% of its activity relative to activity of the composition at the beginning of storage. For example, an active ingredient, e.g., bupivacaine hydrochloride, epinephrine and/or dexamethasone sodium in the formulation does not lose more than about 5% of its activity relative to activity of the composition at the beginning of storage.

In some embodiments, each of bupivacaine hydrochloride, epinephrine and dexamethasone sodium in the syringed formulation have a potency of at least about 90% after storage for about 90 days at a temperature of about 5° C. and a subsequent storage of about 25° C. for a minimum of about 72 hours or any kinetic temperature equivalent storage. The syringed formulation is also substantially free of impurities even after long term storage, i.e., amount of impurities in the syringed formulation remains low, even after long term storage. For example, the syringed formulation does not comprise any impurities exceeding ICH/FDA guidelines after storage for about 90 days at a temperature of about 5° C. and a subsequent storage of about 25° C. for a minimum of about 72 hours or any kinetic temperature equivalent storage. In some embodiments, an impurity in the syringed formulation is below about 1%, below about 0.5%, or below about 0.25% after storage for about 90 days at a temperature of about 5° C. and a subsequent storage of about 25° C. for a minimum of about 72 hours or any kinetic temperature equivalent storage.

In some embodiments, the syringe can be contained in a container. For example, a syringe comprising a formulation described herein can be contained in a light sensitive container. For example, the container containing the syringe can be light-sensitive container having a light transmission of less than less than about 5%, e.g., less than about 4.5%, less than about 4%, less than about 3.5%, less than about 3%, less than about 2.5%, less than about 2%, less than about 1.5% or less than about 1% at any wavelength between about 290 nm and about 450 nm. In some embodiments, the light-sensitive container, containing the syringe, comprises an amber-colored film or an amber-colored plastic.

In some embodiments, the syringe containing the formulation can be in a hermetically sealed package. For example, the syringe containing the formulation is in a hermetically sealed package to protect the syringe contents from oxygen intrusion.

In some embodiments, the container comprising the formulation, e.g., the syringe comprising the formulation can be in a hermetically sealed package. For example, the container comprising the formulation (i.e., the formulation container) is in a hermetically sealed package to protect one of more components of the formulation from oxygen intrusion.

Generally, the interior environment of the package comprising the formulation container has reduced oxygen, i.e., O₂ level. For example, the interior environment of the package comprises less than 10% (v/v) oxygen. In some embodiments, the interior environment of the package has about 5% (v/v) or lower, about 4% (v/v) or lower, about 3% (v/v) or lower, about 2.5% (v/v) or lower, about 2% (v/v) or lower, about 1.5% (v/v) or lower, about 1% (v/v) or lower, about 0.75% (v/v) or lower, about 0.5% (v/v) or lower, about 0.25% (v/v) or lower, about 0.2% (v/v) or lower, about 0.15% (v/v) or lower, about 0.1% (v/v) or lower, about 0.055% (v/v) or lower, or about 0.025% (v/v) or lower oxygen content. In some embodiments, the package comprising the syringe is substantially free of oxygen.

Accordingly, in one aspect provided herein is a package comprising a container comprising a formulation described herein, wherein an interior environment of the package has a reduced oxygen level.

Generally, the syringe is inserted into the package and the environment in the package manipulated to create an environment with reduced oxygen in the package. In some embodiments, the environment with reduced oxygen within the package is created by emptying the environment inside the package through the application of vacuum. After emptying the container, the environment inside the package is an environment with reduced oxygen. Then, the package is sealed, e.g., hermetically sealed.

In some embodiments, the environment with reduced oxygen within the container is created by emptying the environment inside the package through the introduction of a low oxygen gas source and/or an inert gas, e.g., N₂. After introducing the inert gas into the package, the environment inside the package is an environment with reduced oxygen. Then, the package is sealed, e.g., hermetically sealed.

In some embodiments, the environment with reduced oxygen within the container is created through the introduction of an oxygen scavenger. After introducing the oxygen scavenger into the package, the environment inside the package is an environment with reduced oxygen. Then, the package is sealed, e.g., hermetically sealed. Exemplary oxygen scavengers include, but are not limited to, triazines, maleimides, formaldehydes, amines, carboxamides, alkylcarboxyl-azo compounds cumine-peroxide compounds morpholino and amino derivatives morpholine and piperazine derivatives, amine oxides, alkanolamines, aliphatic and aromatic polyamines sulfurous acid or a salt thereof, such as a sulfite, bisulfite, or thiosulfite. It is noted that the oxygen scavenger can be added directly to the package interior or it can be contained a container, e.g., packet, pouch or the like that can be added to the package.

In some embodiment, each of bupivacaine hydrochloride, epinephrine and dexamethasone sodium in the formulation have a potency of at least about 90% after storage for about 90 days at room temperature when stored in a package described herein. The formulation is also substantially free of impurities even after long term storage, i.e., amount of impurities in the formulation remains low, even after long term storage at room temperature. For example, the formulation stored in a package described herein does not comprise any impurities exceeding ICH/FDA guidelines after storage for about 90 days at room temperature. In some embodiments, an impurity in the formulation stored in a package described herein is below about 1%, below about 0.5%, or below about 0.25% after storage for about 90 days at room temperature.

In some embodiments, long-term storage means that formulation is stored either as a liquid or solid at room temperature for at least 90 days.

The invention also provides a container comprising a formulation as described herein and a kit comprising instructions for use.

In another aspect, provided herein is a method for preparing a ready-to-use liquid formulation described herein. Generally, the method comprises optionally adding a preservative or antimicrobial agent (e.g., methylparaben) to a heated, e.g., to about 60-70° C., salt (e.g., sodium chloride) solution in a first container; (b) adding bupivacaine HCl to the heated solution from (a); (c) cooling the heated solution from (b), optionally by adding cold water; (d) adding dexamethasone sodium phosphate to the cooled solution from (c); (e) optionally, transferring the solution from (d) to a new container; (f) adjusting the pH of the solution to a range from about 3.65 to about 3.80; (g) adding an antioxidant agent (e.g., sodium metabisulfite) to a salt (e.g., sodium chloride) solution in a second container; (h) adjusting the pH of the solution from (g) to a range from about 3.5 to about 6.0; (i) adding epinephrine to the pH adjusted solution from (h); (j) mixing the solution comprising epinephrine from (i) with the solution from (f); (k) adjusting the volume of the solution from (j) to the required final volume; (l) adjusting the pH to the desired range, e.g., a pH range from about 3.5 to about 4.1, preferably a pH range from about 3.7 to about 3.9; and (m) optionally, sterile filtering the pH adjusted solution from (l), optionally using a 0.22-micron filter.

It is noted that formulations described herein can be prepared without a preservative. Accordingly, in some embodiments, a method for preparing a ready-to-use liquid formulation described herein does not comprise use of a preservative. Generally, the method comprises obtaining a heated, e.g., to about 60-70° C., salt (e.g., sodium chloride) solution in a first container; (b) adding bupivacaine HCl to the heated solution from (a); (c) cooling the heated solution comprising bupivacaine HCl from (b), optionally by adding cold water; (d) adding dexamethasone sodium phosphate to the cooled solution comprising bupivacaine HCl from (c); (e) optionally, transferring the solution comprising bupivacaine HCl and dexamethasone sodium phosphate from (d) to a new container; (f) adjusting the pH of the solution comprising bupivacaine HCl and dexamethasone sodium phosphate to a range from about 3.65 to about 3.80; (g) adding an antioxidant agent (e.g., sodium metabisulfite) to a salt (e.g., sodium chloride) solution in a second container; (h) adjusting the pH of the solution comprising sodium metabisulfite from (g) to a range from about 3.5 to about 6.0; (i) adding epinephrine to the pH adjusted solution from (h); (j) mixing the solution comprising epinephrine from (i) with the solution comprising bupivacaine HCl and dexamethasone sodium phosphate from (f); (k) adjusting the volume of the solution from (j) to the required final volume; (l) adjusting the pH to the desired range, e.g., a pH range from about 3.5 to about 4.1, preferably a pH range from about 3.7 to about 3.9; and (m) optionally, sterile filtering the pH adjusted solution from (l), optionally using a 0.22-micron filter.

In yet another aspect, provided herein is a method for alleviating post-operative pain in a subject. Generally, the method comprises administering a ready-to-use-liquid formulation to a subject in need thereof. In some embodiments, the subject in need of alleviating post-operative pain is a subject receiving or having received an arthroplasty treatment, e.g., arthroplasty treatment of the knee, hip, or other joint. In some embodiments, the subject in need of alleviating post-operative pain is a subject undergoing or having undergone knee replacement surgery.

In some embodiments, the ready-to-use-liquid formulation described herein is used as a supraclavicular block, which is useful for brachial plexus; arm, hand and shoulder surgeries; and AV Fistulas. In some embodiments, the ready-to-use-liquid formulation described herein is used as an erector spinae block, which is beneficial for block intercostal nerves; thoracic surgeries; Vanfossen breast surgeries; and traumas, rib fractures and back cases. In some embodiments, the ready-to-use-liquid formulation described herein is used as a PEC I block, which can be helpful for lateral & medial pectoral nerves. In some embodiments, the ready-to-use-liquid formulation described herein is used as a PEC II block, which is suitable for intercostal T2-T6 long thoracic and breast surgeries. In some embodiments, the ready-to-use-liquid formulation described herein is used as a transverse abdominal plane (TAP) block, which is useful for surgeries involving abdomen below the umbilicus; lateral ports/incisions, rectus plane block (e.g., abdominal procedures above the umbilicus) and vertical midline incisions. In some embodiments, the ready-to-use-liquid formulation described herein is used as a Fascia Iliaca (FI) block, which is beneficial for hip fractures and anteromedial knee, and femoral head fractures and total hip surgeries. In some embodiments, the ready-to-use-liquid formulation described herein is used as an adductor canal block or saphenous block, which are useful for knee and total knee surgeries. For example, the ready-to-use-liquid formulation described herein can be used a saphenous block along with a POP (popliteal block) block in lower leg surgeries. In some embodiments, the ready-to-use-liquid formulation described herein is used as Popliteal (POP) Block, which is suitable for anterior/lateral portion of lower extremity, tibia, fibula, ankle, and foot surgeries.

Exemplary embodiments of the various aspects described herein can be described by one or more of the following numbered embodiments:

Embodiment 1: A ready-to-use liquid formulation comprising: bupivacaine hydrochloride in an amount from about 3.25 mg/mL to about 4.25 mg/mL; epinephrine hydrochloride in an amount from about 4.5 mcg/mL to about 5.5 mcg/mL, based on an epinephrine free base; dexamethasone sodium phosphate in an amount from about 0.05 mg/mL to about 0.15 mg/mL; a salt in an amount of about 7.7 mg/mL to about 8.7 mg/mL; optionally, an antioxidant in an amount up to about 23 mcg/mL; optionally, a preservative in an amount up to about 0.85 mg/mL; a sufficient amount of sterile water for injection; and a sufficient amount of a pH adjusting agent to obtain a pH of from about 3.2 to about 4.4.

Embodiment 2: The ready-to-use liquid formulation of Embodiment 1, wherein the pH is from about 3.7 to about 3.9.

Embodiment 3: The ready-to-use liquid formulation of any one of Embodiments 1-2, wherein the salt is selected from the group consisting of sodium chloride, potassium chloride, magnesium chloride, calcium chloride, and potassium phosphate.

Embodiment 4: The ready-to-use liquid formulation of any one of Embodiments 1-3, wherein the salt is sodium chloride.

Embodiment 5: The ready-to-use liquid formulation of any one of Embodiments 1-4, wherein the antioxidant is selected from the group consisting of sodium metabisulfite, sodium sulfite, sodium bisulfite, sodium thiosulfate, vitamin C (ascorbic acid), vitamin E, vitamin A, B vitamins (e.g. vitamin B₆), flavonoids, selenium, carotenoids (e.g. beta-carotene). Additional exemplary antioxidants include propyl, octyl and dodecyl esters of gallic acid, butylated hydroxyanisole (BHA), butylated hydroxytoluene (BHT), nordihydroguairetic acid, and alkylated parabens.

Embodiment 6: The ready-to-use liquid formulation of any one of Embodiments 1-5, wherein the antioxidant is sodium metabisulfite.

Embodiment 7: The ready-to-use liquid formulation of any one of Embodiments 1-6, wherein the pH adjusting agent is selected from the group consisting of organic acids, mineral acids, organic bases, hydroxides of alkaline metals and alkaline earth metals, and any combinations thereof.

Embodiment 8: The ready-to-use liquid formulation of any one Embodiments 1-7, wherein the pH adjusting agent is hydrochloric acid, sodium hydroxide, or any combination thereof.

Embodiment 9: The ready-to-use liquid formulation of any one of Embodiments 1-8, wherein the preservative is selected from the group consisting of antimicrobial preservatives, antifungal preservatives, antiprotozoan preservatives, alcohol preservatives, acidic preservatives, and other preservatives.

Embodiment 10: The ready-to-use liquid formulation of any one of Embodiments 1-9, wherein the preservative is methylparaben.

Embodiment 11: The ready-to-use liquid formulation of any one of Embodiments 1-10, wherein the formulation is substantially free of antimicrobial preservatives.

Embodiment 12: The ready-to-use liquid formulation of any one of Embodiments 1-11, wherein the formulation comprises: bupivacaine hydrochloride in an amount of about 3.75 mg/mL; epinephrine hydrochloride in an amount of about 5 mcg/mL, based on an epinephrine free base; dexamethasone sodium phosphate in an amount of about 0.1 mg/mL; a salt in an amount of about 8.2 mg/mL; sodium metabisulfite in an amount of about 23 mcg/mL; optionally methylparaben in an amount of 0.75 mg/mL; a sufficient amount of sterile water for injection; and a sufficient amount of a pH adjusting agent to obtain a pH of from about 3.7 to about 3.9.

Embodiment 13: The ready-to-use liquid formulation of any one of Embodiments 1-12 further comprising a second preservative.

Embodiment 14: The ready-to-use liquid formulation of any one of Embodiments 1-13 further comprising a second preservative selected from the group consisting of benzyl alcohol, propylparaben, and any combination thereof.

Embodiment 15: The ready-to-use liquid formulation of any one of Embodiments 1-14, wherein each of bupivacaine hydrochloride, epinephrine hydrochloride and dexamethasone sodium have a potency of at least about 90% after storage for about 90 days at a temperature of about 5° C. and a subsequent storage of 25° C. for a minimum of about 72 hours or any kinetic temperature equivalent storage.

Embodiment 16: The ready-to-use liquid formulation of any one of Embodiments 1-15, wherein the ready-to-use liquid formulation does not comprise an impurity exceeding ICH/FDA guidelines after storage for about 90 days at a temperature of about 5° C. and a subsequent storage of about 25° C. for a minimum of about 72 hours or any kinetic temperature equivalent storage.

Embodiment 17: A syringe comprising the ready-to-use liquid formulation of any one of Embodiments 1-16.

Embodiment 18: A syringe comprising about 35 mL of the ready-to-use liquid formulation of any one of Embodiments 1-16.

Embodiment 19: The syringe of any one of Embodiments 17-18, wherein the syringe is in a light-sensitive container.

Embodiment 20: The syringe of any one of Embodiments 17-19, wherein the syringe is in a light-sensitive container and wherein the light-sensitive container has a light transmission of less than about 5% at any wavelength between about 290 nm and about 450 nm.

Embodiment 21: The syringe of any one of Embodiments 17-20, wherein the syringe is in a hermetically sealed package.

Embodiment 22: The syringe of any one of Embodiments 17-21, wherein each of bupivacaine hydrochloride, epinephrine hydrochloride and dexamethasone sodium have a potency of at least about 90% after storage for about 90 days at a temperature of about 5° C. and a subsequent storage of 25° C. for a minimum of about 72 hours or any kinetic temperature equivalent storage.

Embodiment 23: The syringe of any one of Embodiments 17-22, wherein the ready-to-use liquid formulation substantially does not comprise an impurity exceeding ICH/FDA guidelines after storage for about 90 days at a temperature of about 5° C. and a subsequent storage of about 25° C. for a minimum of about 72 hours or any kinetic temperature equivalent storage.

Embodiment 24: A container comprising the ready-to-use liquid formulation of any one of the Embodiments 1-16

Embodiment 25: The container of Embodiment, 24, wherein the container is a light-sensitive container having a light transmission of less than about 5% at any wavelength between about 290 nm and about 450 nm.

Embodiment 26: The container of any one of Embodiments 24-25, wherein the container is hermetically sealed.

Embodiment 27: The container of any one of Embodiments 24-26, wherein each of bupivacaine hydrochloride, epinephrine hydrochloride and dexamethasone sodium have a potency of at least about 90% after storage for about 90 days at a temperature of about 5° C. and a subsequent storage of about 25° C. for a minimum of about 72 hours or any kinetic temperature equivalent storage.

Embodiment 28: The container of any one of Embodiments 24-27, wherein the ready-to-use liquid formulation does not comprise an impurity exceeding ICH/FDA guidelines after storage for about 90 days at a temperature of about 5° C. and a subsequent storage of about 25° C. for a minimum of about 72 hours or any kinetic temperature equivalent storage.

Embodiment 29: A process for preparing the ready-to-use liquid formulation of Embodiments 1-16, the process comprising: (a) optionally, adding a preservative to a heated salt solution in a first container; (b) adding bupivacaine hydrochloride to the heated solution, optionally comprising the methylparaben, from (a); (c) cooling the heated solution from (b), optionally by adding cold water; (d) adding dexamethasone sodium phosphate to the cooled solution from (c); (e) optionally, transferring the solution from (d) to a new container; (f) adjusting the pH of the solution from (e) to a range from about 3.65 to about 3.80; (g) adding an antioxidant to a salt solution in a second container; (h) adjusting the pH of the solution comprising the antioxidant from (g) to a range from about 3.5 to about 6.0; (i) adding epinephrine to the pH adjusted solution from (h); (j) mixing the solution from (i) with the solution from (f); (k) adjusting the volume of the solution from (j) to the required final volume; (l) adjusting the pH of solution from (j) to a range from about 3.7 to about 3.9; and (m) optionally, sterile filtering the pH adjusted solution from (l), optionally using a 0.22-micron filter.

Embodiment 30: A method for alleviating post-operative pain in a subject, the method comprising administering a ready-to-use-liquid formulation of any one of Embodiments 1-16.

Embodiment 31: A package comprising a container, wherein the container comprises the ready-to-use liquid formulation of any one of Embodiments 1-16, and wherein an interior environment of the package has a reduced oxygen level.

Embodiment 32: The package of Embodiment 31, wherein the interior environment of the package comprises an oxygen scavenger and/or an inert gas.

Embodiment 33: The package of any one of Embodiments 31-32, wherein at least one of bupivacaine hydrochloride, epinephrine hydrochloride and dexamethasone sodium have a potency of at least about 90% after storage for about 90 days at room temperature.

Embodiment 34: The package of any one of Embodiments 31-33, wherein each of bupivacaine hydrochloride, epinephrine hydrochloride and dexamethasone sodium have a potency of at least about 90% after storage for about 90 days at room temperature.

Embodiment 35: The package of any one of Embodiments 31-34, wherein the ready-to-use liquid formulation does not comprise an impurity exceeding ICH/FDA guidelines after storage for about 90 days at room temperature.

Embodiment 36: The package of any one of Embodiments 31-35, wherein the package is hermetically sealed.

Embodiment 37: The package of any one of Embodiments 31-36, wherein the package is a light-sensitive package.

Embodiment 38: The package of any one of Embodiments 31-37, wherein the light-sensitive package has a light transmission of less than about 5% at any wavelength between about 290 nm and about 450 nm.

Embodiment 39: The package of any one of Embodiments 31-38, wherein the container comprising the ready-to-use liquid formulation is a syringe of any one of Embodiments 17-23 or a container of any one of Embodiments 24-28.

Embodiment 40: The package of any one of Embodiments 31-39, further comprising instructions for using the ready-to-use liquid formulation.

### Some Selected Definitions

For convenience, the meaning of some terms and phrases used in the specification, examples, and appended claims, are provided below. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects provided herein, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those provided herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±5% (e.g., ±4%, ±3%, ±2%, ±1%) of the value being referred to.

Where a range of values is provided, each numerical value between the upper and lower limits of the range is contemplated and disclosed herein.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

The description of embodiments of the disclosure is not intended to be exhaustive or to limit the disclosure to the precise form disclosed. While specific embodiments of, and examples for, the disclosure are disclosed herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments disclosed herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples in no way should be construed as being further limiting.

### EXAMPLES

### Example 1

### Preparation of an Exemplary Ready-To-Use Liquid Formulation of the Invention

Prepare a bupivacaine HCl+dexamethasone sodium phosphate formulation as follows: (a) obtain a proper beaker (Beaker 1) and add 18% (18% from the target scale) of the required 0.9% sodium chloride. Heat the 0.9% sodium chloride solution to ˜60-70° C.; (b) weigh the appropriate amount of methylparaben and add it into the heated 0.9% sodium chloride beaker (Beaker 1); (c) weigh the appropriate amount of bupivacaine HCl and add it to the beaker (Beaker 1) that contains methylparaben and 0.9% sodium chloride solution; (d) bring down the temperature of bupivacaine HCl+methylparaben+0.9% sodium chloride solution by adding refrigerated sterile water; (e) weight the required quantity of dexamethasone sodium phosphate and add into the same beaker (Beaker 1); (f) add 16% of the targeted sterile water for injection (SWFI), and Beaker 1 content into a pool bag or suitable container and fill the rest of the pool bag with 0.9% sodium chloride up to 90% of the full target scale; (g) mix the content; and (h) adjust the in-process pH to 3.65-3.80.

Prepare an epinephrine formulation as follows: (a) obtain a new beaker (Beaker 2) and add 5% of the required (target) 0.9% sodium chloride into the beaker; (b) weigh the required amount of sodium metabisulfite (SMBS) and transfer it into the beaker (Beaker 2) (c) adjust the in-process pH to 3.50-6.00; (d) weigh the required amount of epinephrine and add to Beaker 2 containing 0.9% sodium chloride and sodium metabisulfite; and (e) mix until content is completely dissolved.

Transfer Beaker 2 content into the same pool bag that contains bupivacaine HCl, dexamethasone sodium phosphate and methylparaben. QS the bag to its final volume with 0.9% sodium chloride. Perform a final pH check. Adjust as necessary to target pH range 3.70-3.90. Filter the solution through a 0.22-micron filter.

### Example 2

### Potency and Stability of Exemplary Formulations

In this study, inventors tested the potency and stability of some exemplary formulations comprising Bupivacaine HCI 0.375%, Dexamethasone Phosphate 0.01%, Epinephrine 1:200,000 (5 pg/mL) and 27.5 mg/ml of a preservative (methylparaban). An exemplary tested formulation is summarized in Table 1.

TABLE 1
Exemplary formulation

| Finished Product | Bupivacaine HCI 0.375% and Dexamethasone Phosphate | |
|---|---|---|
| Description: | 0.01% with Epinephrine 1:200,000 (5 pg/mL), 27.5 mg/ml | |
| | methylparaban, 30 mL Total Volume | |
| Route(s) of Administration: | For Nerve Block Only | |
| Labeled Packaging: | 30 mL in a 35 mL syringe | |
| Fill Volume: | 30 mL | |
| Dispensing Device | Luer Lock Cap | |
| (Primary Packaging)/SP#: | | |
| Secondary Packaging/SP#: | White 8″ × 8.25″ × 3.5″ 10-Hole Box (9051) | |
| Storage Requirements: | Long-Term Storage | Accelerated Condition |
| | Refrigerated (5° C. ± 3° C.), | Room Temperature (25° C. ± 2° C. |
| | Horizontal Orientation, | and 40% RH ± 5% RH) |
| | Protected from Light | Horizontal Orientation, |
| | | Protected from Light |

The samples were produced using an approved batch record and shipped to contract laboratories for stability testing. The samples were stored according to ICH Q1A (R2) as follows:
Long term: Refrigerated (5° C. ±3° C.) in Horizontal Orientation, Protected from Light
Accelerated Condition: Room Temperature (25° C. ±2° C. and 40% RH ±5% RH) Horizontal Orientation, Protected from Light

The method development procedure for the strength and potency aligned with the defined criteria in USP <1225> for a stability indicating method. The procedures used for microbiology testing were based on method suitability performed at T0.

The testing was completed by contract laboratories in compliance with the stability protocol of Bupivacaine HCI 0.375% and Dexamethasone Phosphate 0.01% with Epinephrine 1:200,000 (5 pg/mL) Injection. The test methods and acceptance criteria used are summarized in Table 2.

TABLE 2
Test methods and Acceptance Criteria

| Test Attribute | Test Method | Acceptance Criteria | |
|---|---|---|---|
| Appearance | Visual, USP <790> | Clear, colorless, and essentially free from | |
| | | visible particulates | |
| pH | USP <791> | Between 3.3 and 6.0 | |
| Particulate Matter | USP <788> | ≥10 pm | NMT 6000 per container |
| | | ≥25 pm | NMT 600 per container |

| | | |
|---|---|---|
| Identification | UPLC/UV method | The retention time of the major peak of the |
| | | sample solution corresponds to that of the |
| | | standard solution |
| Strength/Potency- | | Potency is between 90.0% and 110.0% of |
| Bupivacaine HCI | | the labeled amount of Bupivacaine HCI |
| Strength/Potency- | | Potency is between 90.0% and NMT |
| Dexamethasone | | 110.0% of the labeled amount of |
| Phosphate | | Dexamethasone Phosphate, present as the |
| | | disodium salt |
| Strength/Potency- | | Potency is between 90.0% and 115.0% of |
| Epinephrine | | the labeled amount of Epinephrine |
| Impurities | ICH Q3B(R2) | Extraneous peaks ≥ 0.1% of parent are |
| | Impurities | reported |
| | in New Drug Products | and evaluated |
| Osmolality | USP <785> | For information only |
| Sterility | USP <71>, USP | No growth of test products |
| | <1223> | |
| Bacterial Endotoxin | USP <85> | NMT 5.8 EU/mL |
| Container Closure | USP <1207> | Passes |
| Integrity Testing (CCIT) | | |

An overview of the time points, conditions and results is summarized in Table 3.

TABLE 3
Overview of results

| Lot | | | |
|---|---|---|---|
| Number | Time Point Testing Required¹ | Test Attribute | Results |
| 2030474 | T0, T30, T47, T61, T61 + 48 hr^{A}, T90, T90 + | Appearance | Conforms |
| 2030475 | 48 hr^{A} | | |
| 2030476 | T0, T30, T47, T61, T61 + 48 hr^{A}, T90, T90 + | pH | Conforms |
| | 48 hr^{A} | | |
| | T0, T30, T47, T61, T61 + 48 hr^{A}, T90, T90 + | Particulate Matter | Conforms |
| | 48 hr^{A} | | |
| | T0, T30, T47, T61, T61 + 48 hr^{A}, T90, T90 + | Identification | Conforms |
| | 48 hr^{A} | | |
| | T0, T30, T47, T47 + 48 hr^{A}, T61, T61 + 24 hr^{A}, | Strength/Potency | Conforms |
| | T61 + 48 hr^{A}, T90, T90 + 48 hr^{A} | | |
| | T0, T30, T47, T47 + 48 hr^{A}, T61, T61 + 24 hr^{A}, | Impurities | Conforms |
| | T61 + 48 hr^{A}, T90, T90 + 48 hr^{A} | | |
| | T30, T60, T90 | CCIT | Conforms |

^{A}Accelerated condition: Room temperature (25° C. ± 2° C. and 40% RH ± 5% RH), protected from light.

### Impurity/Potency Assessment

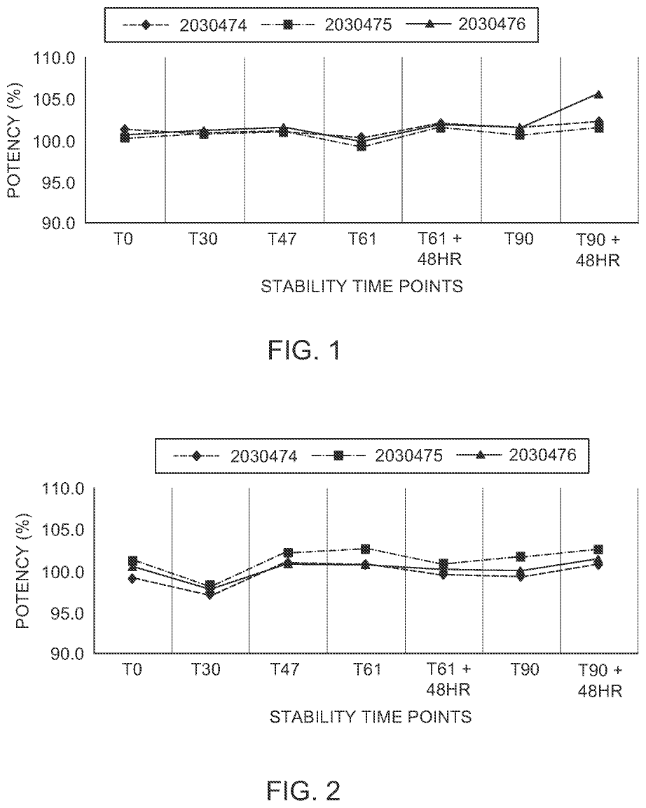

FIG. 1 shows the potency trend of Bupivacaine HCl 0.375% in an exemplary formulation of the disclosure. All values met the acceptance criteria of NLT 90.0 to NMT 110.0% for Bupivacaine HCl 0.375%. All impurities met FDA/ICH Guidelines.

FIG. 2 shows the potency trend of Dexamethasone Phosphate 0.01% in an exemplary formulation of the disclosure. All values met the acceptance criteria of NLT 90.0 to NMT 110.0% for Dexamethasone Phosphate 0.01%. All impurities met FDA/ICH Guidelines.

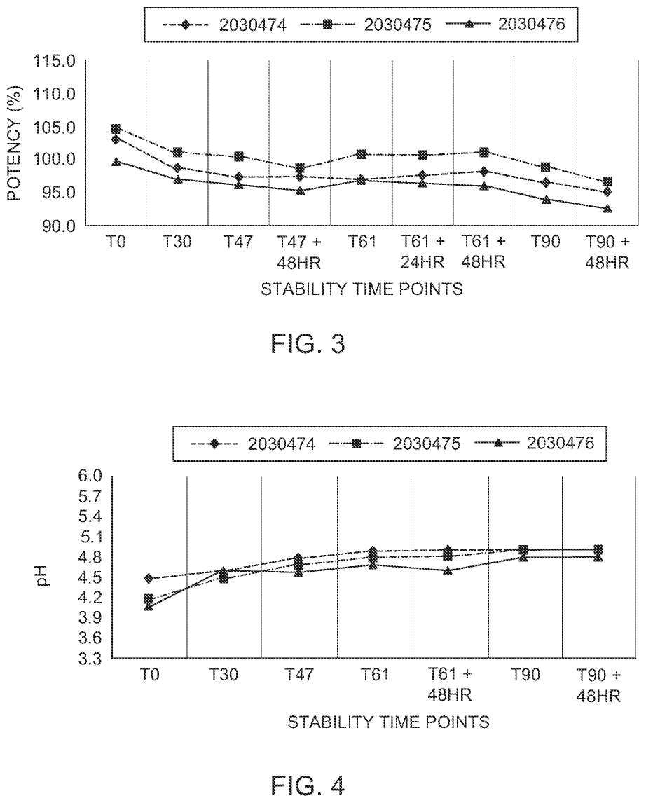

FIG. 3 shows the potency trend of Epinephrine 1:200,000 in an exemplary formulation of the disclosure. All values met the acceptance criteria of NLT 90.0 to NMT 115.0% for Epinephrine 1:200,000. All impurities met FDA/ICH Guidelines.

FIG. 4 shows the pH trend of Bupivacaine HCl 0.375%, Dexamethasone Phosphate 0.01% and Epinephrine 1:200,000 in an exemplary formulation of the disclosure. All values met the acceptance criteria of 3.3-6.0.

The data presented herein show that defined acceptance criteria of stability protocol were met for all three (3) batches tested, with passing results received for each test performed. Additionally, Bupivicaine, Dexamethasone Phosphate and Epinephrine impurities were evaluated and met the acceptable limits established by ICH guidelines, FDA, and in-house SOP.

The stability test results and data presented herein demonstrate that Bupivacaine HCl 0.375% and Dexamethasone Phosphate 0.01% with Epinephrine 1:200,000 (5 pg/mL) Injection manufactured using the same material of construction with a batch size <100 L is stable for 90 days under refrigerated condition (5° C. ±3° C.) along with 48 hr at controlled room temperature, protected from light and freezing.

### Example 3

### Potency and Stability of Exemplary Formulations Prepared without a Preservative

In this study, inventors tested the potency and stability of some exemplary formulations comprising Bupivacaine HCI 0.375% and Dexamethasone Phosphate 0.01% with Epinephrine 1:200,000 (5 μg/mL) but no preservative(s).

The formulations without a preservative were prepared following the method described in Example 1 except no preservative(s) was used.

An exemplary tested formulation prepared without a preservative(s) is summarized in Table 4.

TABLE 4
Exemplary formulation

| Finished Product | Bupivacaine HCI 0.375% and Dexamethasone Phosphate | |
|---|---|---|
| Description: | 0.01% with Epinephrine 1:200,000 (5 μg/mL), 30 mL Total | |
| | Volume (no preservative(s)) | |
| Route(s) of Administration: | For Nerve Block Only | |
| Labeled Packaging: | 30 mL in a 35 mL syringe | |
| Fill Volume: | 30 mL | |
| Dispensing Device | Luer Lock Cap | |
| (Primary Packaging)/SP#: | | |
| Secondary Packaging/SP#: | White 8″ × 8.25″ × 3.5″ 10-Hole Box | |
| Storage Requirements: | Long-Term Storage | Accelerated Condition |
| | Refrigerated (5° C. ± 3° C.), | Room Temperature (25° C. ± 2° C. |
| | Horizontal Orientation, | and 40% RH ± 5% RH) |
| | Protected from Light | Horizontal Orientation, |
| | | Protected from Light |

The samples were produced using an approved batch record and shipped to contract laboratories for stability testing. The samples were stored according to ICH Q1A (R2) as follows:
Long term: Refrigerated (5° C. ±3° C.) in Horizontal Orientation, Protected from Light
Accelerated Condition: Room Temperature (25° C. ±2° C. and 40% RH ±5% RH) Horizontal Orientation, Protected from Light

The method development procedure for the strength and potency aligned with the defined criteria in USP <1225> for a stability indicating method. The procedures used for microbiology testing were based on method suitability performed at T0.

The testing was completed by contract laboratories in compliance with the stability protocol of Bupivacaine HCI 0.375% and Dexamethasone Phosphate 0.01% with Epinephrine 1:200,000 (5 pg/mL) Injection. The test methods and acceptance criteria used are summarized in Table 5.

TABLE 5
Test methods and Acceptance Criteria

| Test Attribute | Test Method | Acceptance Criteria | |
|---|---|---|---|
| Appearance | Visual, USP <790> | Clear, colorless, and essentially free from | |
| | | visible particulates | |
| pH | USP <791> | Between 3.3 and 6.0 | |
| Particulate Matter | USP <788> | ≥10 pm | NMT 6000 per container |
| | | ≥25 pm | NMT 600 per container |

| | | |
|---|---|---|
| Identification | UPLC/UV method | The retention time of the major peak of the |
| | | sample solution corresponds to that of the |
| | | standard solution |
| Strength/Potency- | | Potency is between 90.0% and 110.0% of |
| Bupivacaine HCI | | the labeled amount of Bupivacaine HCI |
| Strength/Potency- | | Potency is between 90.0% and NMT |
| Dexamethasone | | 110.0% of the labeled amount of |
| Phosphate | | Dexamethasone Phosphate, present as the |
| | | disodium salt |
| Strength/Potency- | | Potency is between 90.0% and 115.0% of |
| Epinephrine | | the labeled amount of Epinephrine |
| Impurities | ICH Q3B(R2) | Extraneous peaks ≥ 0.1% of parent are |
| | Impurities | reported |
| | in New Drug Products | and evaluated |
| Osmolality | USP <785> | For information only |
| Sterility | USP <71>, USP | No growth of test products |
| | <1223> | |
| Bacterial Endotoxin | USP <85> | NMT 5.8 EU/mL |
| Container Closure | USP <1207> | No dye egress |
| Integrity Testing (CCIT) | | |

An overview of the time points, conditions and results is summarized in Table 6.

TABLE 6
Overview of results

| Lot | | | |
|---|---|---|---|
| Number | Time Point Testing Required¹ | Test Attribute | Results |
| 2130289 | T0, T31, T75, T89, T89 + 48 hr^{A}, T122, T122 + | Appearance | Conforms |
| | 48 hr^{A} | | |
| | T0, T31, T75, T89, T89 + 48 hr^{A}, T122, T122 + | pH | Conforms |
| | 48 hr^{A} | | |
| | T0, T31, T75, T89, T89 + 48 hr^{A}, T122, T122 + | Particulate Matter | Conforms |
| | 48 hr^{A} | | |
| | T0, T31, T75, T89, T89 + 48 hr^{A}, T122, T122 + | Identification | Conforms |
| | 48 hr^{A} | | |
| | T0, T31, T75, T89, T89 + 48 hr^{A}, T122, T122 + | Strength/Potency | Conforms |
| | 48 hr^{A} | | |
| | T0, T31, T75, T89, T89 + 48 hr^{A}, T122, T122 + | Impurities | Conforms |
| | 48 hr^{A} | | |
| | T90, T120 | CCIT | Conforms |

^{A}Accelerated condition: Room temperature (25° C. ± 2° C. and 40% RH ± 5% RH), protected from light.

The data presented herein show that defined acceptance criteria of stability protocol were met for the tested exemplary formulation without any preservative(s), with passing results received for each test performed. Additionally, Bupivicaine, Dexamethasone Phosphate and Epinephrine impurities were evaluated and met the acceptable limits established by ICH guidelines, FDA, and in-house SOP.

The stability test results and data presented herein demonstrate that Bupivacaine HCl 0.375% and Dexamethasone Phosphate 0.01% with Epinephrine 1:200,000 (5 pg/mL) Injection manufactured using the same material of construction with a batch size <100 L but without preservatives is stable for 90 days under refrigerated condition (5° C. ±3° C.) along with 48hr at controlled room temperature, protected from light and freezing.

### Example 4

### Potency and Stability of Exemplary Formulations Stored at Room Temperature

A preliminary evaluation of the feasibility of packaging syringes was performed. Hermetically sealed pouch containing a syringe with the exemplary formulation from Table 4 was flushed with nitrogen to reduce oxygen levels. The result of testing after storage for 90 days at room temperature are shown in Table 7.

TABLE 7
Potency (% label claim) after storage at room temperature for 90 days

| | Potency |
|---|---|
| Dexamethasone | 95.9% |
| Bupivacaine | 97.2% |
| Epinephrine | 104.2% |

All patents and other publications; including literature references, issued patents, published patent applications, and co-pending patent applications; cited throughout this application are expressly incorporated herein by reference for the purpose of providing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A ready-to-use liquid formulation comprising:
bupivacaine hydrochloride in an amount from about 3.25 mg/mL to about 4.25 mg/mL;
epinephrine hydrochloride in an amount from about 4.5 mcg/mL to about 5.5 mcg/mL, based on an epinephrine free base;
dexamethasone sodium phosphate in an amount from about 0.05 mg/mL to about 0.15 mg/mL;
a salt in an amount of about 7.7 mg/mL to about 8.7 mg/mL;
optionally, an antioxidant in an amount up to about 23 mcg/mL;
optionally, a preservative in an amount up to about 0.85 mg/mL;
a sufficient amount of sterile water for injection; and
a sufficient amount of a pH adjusting agent to obtain a pH of from about 3.2 to about 4.4.

2. The ready-to-use liquid formulation of claim 1, wherein the pH is from about 3.7 to about 3.9.

3. The ready-to-use liquid formulation of claim 1, wherein the salt is selected from the group consisting of sodium chloride, potassium chloride, magnesium chloride, calcium chloride, and potassium phosphate.

4. The ready-to-use liquid formulation of claim 3, wherein the salt is sodium chloride.

5. The ready-to-use liquid formulation of claim 1, wherein the antioxidant is selected from the group consisting of sodium metabisulfite, sodium sulfite, sodium bisulfite, sodium thiosulfate, vitamin C (ascorbic acid), vitamin E, vitamin A, B vitamins (e.g. vitamin B₆), flavonoids, selenium, carotenoids (e.g. beta-carotene). Additional exemplary antioxidants include propyl, octyl and dodecyl esters of gallic acid, butylated hydroxyanisole (BHA), butylated hydroxytoluene (BHT), nordihydroguairetic acid, and alkylated parabens.

6. The ready-to-use liquid formulation of claim 5, wherein the antioxidant is sodium metabisulfite.

7. The ready-to-use liquid formulation of claim 6, wherein the pH adjusting agent is selected from the group consisting of organic acids, mineral acids, organic bases, hydroxides of alkaline metals and alkaline earth metals, and any combinations thereof.

8. The ready-to-use liquid formulation of claim 7, wherein the pH adjusting agent is hydrochloric acid, sodium hydroxide, or any combination thereof.

9. The ready-to-use liquid formulation of claim 1, wherein the preservative is selected from the group consisting of antimicrobial preservatives, antifungal preservatives, antiprotozoan preservatives, alcohol preservatives, acidic preservatives, and other preservatives.

10. The ready-to-use liquid formulation of claim 9, wherein the preservative is methylparaben.

11. The ready-to-use liquid formulation of claim 1, wherein the formulation is substantially free of antimicrobial preservatives.

12. The ready-to-use liquid formulation of claim 11, wherein the formulation comprises:
bupivacaine hydrochloride in an amount of about 3.75 mg/mL;
epinephrine hydrochloride in an amount of about 5 mcg/mL, based on an epinephrine free base;
dexamethasone sodium phosphate in an amount of about 0.1 mg/mL;
a salt in an amount of about 8.2 mg/mL;
sodium metabisulfite in an amount of about 23 mcg/mL;
optionally methylparaben in an amount of 0.75 mg/mL;
a sufficient amount of sterile water for injection; and
a sufficient amount of a pH adjusting agent to obtain a pH of from about 3.7 to about 3.9.

13. The ready-to-use liquid formulation of claim 1, wherein the formulation further comprises a second preservative.

14. The ready-to-use liquid formulation of claim 13, wherein the second preservative selected from the group consisting of benzyl alcohol, propylparaben, and any combination thereof.

15. The ready-to-use liquid formulation of claim 1, wherein each of bupivacaine hydrochloride, epinephrine hydrochloride and dexamethasone sodium have a potency of at least about 90% after storage for about 90 days at a temperature of about 5° C. and a subsequent storage of 25° C. for a minimum of about 72 hours or any kinetic temperature equivalent storage.

16. The ready-to-use liquid formulation of claim 1, wherein the ready-to-use liquid formulation does not comprise an impurity exceeding ICH/FDA guidelines after storage for about 90 days at a temperature of about 5° C. and a subsequent storage of about 25° C. for a minimum of about 72 hours or any kinetic temperature equivalent storage.

17. A syringe comprising the ready-to-use liquid formulation of claim 1.

18. A syringe comprising about 35 mL of the ready-to-use liquid formulation of claim 1.

19. The syringe of claim 18, wherein the syringe is in a light-sensitive container.

20. The syringe of claim 18, wherein the syringe is in a light-sensitive container and wherein the light-sensitive container has a light transmission of less than about 5% at any wavelength between about 290 nm and about 450 nm.

21. The syringe of claim 18, wherein the syringe is in a hermetically sealed package.

22. The syringe of claim 18, wherein each of bupivacaine hydrochloride, epinephrine hydrochloride and dexamethasone sodium have a potency of at least about 90% after storage for about 90 days at a temperature of about 5° C. and a subsequent storage of 25° C. for a minimum of about 72 hours or any kinetic temperature equivalent storage.

23. The syringe of claim 18, wherein the ready-to-use liquid formulation substantially does not comprise an impurity exceeding ICH/FDA guidelines after storage for about 90 days at a temperature of about 5° C. and a subsequent storage of about 25° C. for a minimum of about 72 hours or any kinetic temperature equivalent storage.

24. A container comprising the ready-to-use liquid formulation of claim 1.

25. The container of claim 24, wherein the container is a light-sensitive container having a light transmission of less than about 5% at any wavelength between about 290 nm and about 450 nm.

26. The container of claim 24, wherein the container is hermetically sealed.

27. The container of claim 24, wherein each of bupivacaine hydrochloride, epinephrine hydrochloride and dexamethasone sodium have a potency of at least about 90% after storage for about 90 days at a temperature of about 5° C. and a subsequent storage of about 25° C. for a minimum of about 72 hours or any kinetic temperature equivalent storage.

28. The container of claim 24, wherein the ready-to-use liquid formulation does not comprise an impurity exceeding ICH/FDA guidelines after storage for about 90 days at a temperature of about 5° C. and a subsequent storage of about 25° C. for a minimum of about 72 hours or any kinetic temperature equivalent storage.

29. A process for preparing the ready-to-use liquid formulation of claim 1, the process comprising:
a. optionally, adding a preservative to a heated salt solution in a first container;
b. adding bupivacaine hydrochloride to the heated solution, optionally comprising the methylparaben, from (a);
c. cooling the heated solution from (b), optionally by adding cold water;
d. adding dexamethasone sodium phosphate to the cooled solution from (c);
e. optionally, transferring the solution from (d) to a new container;
f. adjusting the pH of the solution from (e) to a range from about 3.65 to about 3.80;
g. adding an antioxidant to a salt solution in a second container;
h. adjusting the pH of the solution comprising the antioxidant from (g) to a range from about 3.5 to about 6.0;
i. adding epinephrine to the pH adjusted solution from (h);
j. mixing the solution from (i) with the solution from (f);
k. adjusting the volume of the solution from (j) to the required final volume;
l. adjusting the pH of solution from (j) to a range from about 3.7 to about 3.9; and
m. optionally, sterile filtering the pH adjusted solution from (l), optionally using a 0.22-micron filter.

30. A method for alleviating post-operative pain in a subject, the method comprising administering a ready-to-use-liquid formulation of claim 1.

31. A package comprising a container, wherein the container comprises the ready-to-use liquid formulation of claim 1, and wherein an interior environment of the package has a reduced oxygen level.

32. The package of claim 31, wherein the interior environment of the package comprises an oxygen scavenger and/or an inert gas.

33. The package of claim 31, wherein at least one of bupivacaine hydrochloride, epinephrine hydrochloride and dexamethasone sodium have a potency of at least about 90% after storage for about 90 days at room temperature.

34. The package of claim 33, wherein each of bupivacaine hydrochloride, epinephrine hydrochloride and dexamethasone sodium have a potency of at least about 90% after storage for about 90 days at room temperature.

35. The package of claim 31, wherein the ready-to-use liquid formulation does not comprise an impurity exceeding ICH/FDA guidelines after storage for about 90 days at room temperature.
